(19) United States
(12) Patent Application Publication
(10) Pub. No.: US 2026/0216451 A1
(43) Pub. Date: Jul. 30, 2026

(54) MEDICAL DEVICE, EXTRACORPOREAL UNIT, AND POWER TRANSMISSION SHEET

(71) Applicant: FURUKAWA ELECTRIC CO., LTD., Tokyo (JP)

(72) Inventors: Tsunenori ARAI, Tokyo (JP); Yoshiki NOMURA, Tokyo (JP); Makoto OCHIAI, Tokyo (JP); Junichi HASEGAWA, Tokyo (JP)

(73) Assignee: FURUKAWA ELECTRIC CO., LTD., Tokyo (JP)

(21) Appl. No.: 19/144,239

(22) PCT Filed: Dec. 27, 2023
(86) PCT No.: PCT/JP2023/046895

§ 371 Date: Jun. 27, 2025

(30) Foreign Application Priority Data
Dec. 28, 2022 (JP) 2022-212642

(51) Int. Cl. A61M 5/42; A61M 39/02

(52) U.S. Cl. CPC A61M 5/427; A61M 39/0208; A61M 2039/0238

(57) ABSTRACT

A medical device includes an extracorporeal unit including a power transmission unit having a second coil noncontactly transmitting electric power; a power receiving unit having a first coil receiving the power; an implantable medical instrument in a living body including the power receiving unit; and a notification unit provided to at least one of the extracorporeal unit and the medical instrument, and in a case of a relative relationship between the power transmission unit and the power receiving unit reaching a predetermined state by movement of the power transmission unit, notifying of having entered said predetermined state. The power transmission unit includes a sheet portion, a cable supplying electric power to the second coil, and one or more holding portions for holding the power transmission unit. The second coil is provided inside the sheet portion, and a hole for inserting an injection needle is provided in the second coil.

### TECHNICAL FIELD

The present disclosure relates to a medical device, an extracorporeal unit and a power transmission sheet.

### BACKGROUND ART

In order to inject a medicinal solution into a living body, a body-implantable medical device is used in which a medical instrument is embedded in the living body. This medical device reduces the burden on a patient who has to be injected frequently for the injection of the medicinal solution. The medical instrument of the medical device includes a soft portion through which an injection needle is inserted into the main body portion. Then, the medicinal solution is injected into a medicinal solution container of the medical instrument through the soft portion. The medicinal solution is transported to the blood vessel through a catheter.

With such a medical instrument, since the main body portion is implanted in the living body, it is difficult to identify the position of the soft portion by visual recognition from outside of the living body.

Therefore, using the technology proposed in Patent Document 1, a medical device has been considered that enables easy confirmation of a predetermined location on the implantable medical instrument for use in a living body.

### CITATION LIST

### Patent Document

Patent Document 1: Japanese Unexamined Patent Application, Publication No. 2021-29935

### DISCLOSURE OF THE INVENTION

### Problems to be Solved by the Invention

With the above described such medical device, in order to identify the position of the medical instrument implanted in a living body from outside the living body, an extracorporeal unit used outside the living body has been employed. However, although an implantable medical instrument for use in a living body is mainly considered in Patent Document 1, there is room for improvement in the extracorporeal unit used outside the living body.

An object of the present disclosure is to provide an extracorporeal unit which can reduce the burden on a living body upon medicinal solution injection to a medical instrument inside the living body, a medical device including the extracorporeal unit, and a power transmission sheet constituting the extracorporeal unit.

### Means for Solving the Problems

[1] A medical device includes: an extracorporeal unit including a power transmission unit having a second coil that transmits electric power in a non-contact manner; a power receiving unit having a first coil that receives the electric power transmitted from the power transmission unit; an implantable medical instrument for use in a living body, the implantable medical instrument including the power receiving unit; and a notification unit that is provided to at least either one of the extracorporeal unit and the implantable medical instrument, and in a case of a relative positional relationship between the power transmission unit and the power receiving unit reaching a predetermined state along with movement of the power transmission unit, notifies of having entered said predetermined state, in which the power transmission unit includes a sheet portion of plate shape, a cable supplying electric power to the second coil, and one or more holding portions for holding the power transmission unit, and the second coil is provided inside of the sheet portion, and a hole for insertion of an injection needle is provided in the second coil.

[2] In the medical device as described in [1] above, the holding portion is provided to at least either of the sheet portion and the cable.

[3] In the medical device as described in [1] or [2] above, the holding portion is provided to one main surface of the sheet portion.

[4] In the medical device as described in any one of [1] to [3] above, the sheet portion further includes a coupling hole that is provided to one main surface of the sheet portion and that couples the sheet portion and the holding portion, and a part of the holding portion is inserted in the coupling hole.

[5] In the medical device as described in [1] or [2] above, the holding portion is provided at an outer edge of the sheet portion.

[6] In the medical device as described in [1] or [2] above, the holding portion is provided at an inner edge of the sheet portion.

[7] In the medical device as described in any one of [1] to [6] above, the holding portion is stood on one main surface of the sheet portion.

[8] In the medical device as described in any one of [1] to [7] above, the holding portion includes a through hole.

[9] In the medical device as described in [1] above, the holding portion is provided between the sheet portion and the cable.

[10] An extracorporeal unit detecting a predetermined location of an implantable medical instrument for use in a living body, the implantable medical instrument including a power receiving unit that receives electric power transmitted from outside in a non-contact manner, the extracorporeal unit including: a power transmission unit including a second coil that transmits electric power in a non-contact manner, in which the power transmission unit includes a sheet portion of plate shape, a cable supplying electric power to the second coil, and one or more holding portions for holding the power transmission unit, and the second coil is provided inside of the sheet portion, and a hole for insertion of an injection needle is provided in the second coil.

[11] A power transmission sheet attachable to and detachable from an extracorporeal unit that detects a predetermined location of an implantable medical instrument for use in a living body, the implantable medical instrument including a power receiving unit that receives electric power transmitted from outside in a non-contact manner, in which the power transmission sheet includes a sheet portion of plate shape, and a second coil that transmits electric power in a non-contact manner is provided inside of the sheet portion, one or more holding portions for holding the sheet portion are provided on an outer side of the sheet portion, and a hole for insertion of an injection needle is provided in the second coil.

### Effects of the Invention

According to the present disclosure, it is possible to provide an extracorporeal unit which can reduce the burden on a living body upon medicinal solution injection to a medical instrument inside the living body, a medical device including the extracorporeal unit, and a power transmission sheet constituting the extracorporeal unit.

### BRIEF DESCRIPTION OF THE DRAWINGS

FIG. 1 is a schematic diagram showing an example of an outline configuration of a medical device according to a first embodiment.

FIG. 2 is a block diagram showing an example of a functional configuration of the medical device according to the first embodiment.

FIG. 3 is a schematic diagram showing an example of an outline configuration of a medical instrument constituting the medical device according to the first embodiment.

FIG. 4 is a schematic diagram showing an example of an outline configuration of an extracorporeal unit constituting the medical device according to the first embodiment.

FIG. 5 is a schematic diagram showing an example of an outline configuration of a power transmission unit constituting the medical device according to the first embodiment.

FIG. 6 is an outline drawing showing a state of puncturing an injection needle to a patient with a medical instrument implanted in their chest.

FIG. 7 is an outline drawing showing a state of puncturing an injection needle to a patient with a medical instrument implanted in their arm.

FIG. 8 is a perspective view showing another example of an outline configuration of the power transmission unit constituting the medical device according to the first embodiment.

FIG. 9 is a perspective view showing another example of a holding portion constituting the medical device according to the first embodiment.

FIG. 10 is a perspective view showing another example of a holding portion constituting the medical device according to the first embodiment.

FIG. 11 is a perspective view showing another example of a holding portion constituting the medical device according to the first embodiment.

FIG. 12 is a perspective view showing another example of a holding portion constituting the medical device according to the first embodiment.

FIG. 13 is a perspective view showing another example of a holding portion constituting the medical device according to the first embodiment.

FIG. 14 is a schematic diagram showing an example of an outline configuration of a medical device according to a second embodiment.

FIG. 15 is a block diagram showing an example of a functional configuration of the medical device according to the second embodiment.

FIG. 16 is a schematic diagram showing an example of an outline configuration of a medical instrument constituting the medical device according to the second embodiment.

FIG. 17 is a schematic diagram showing an example of an outline configuration of an extracorporeal unit constituting the medical device according to the second embodiment.

### PREFERRED MODE FOR CARRYING OUT THE INVENTION

Hereinafter, embodiments of the present invention will be described in detail.

The present inventors have found the following matters as a result of thorough investigation.

First, in the case of performing non-contact power supply from an extracorporeal unit used outside a living body to an implantable medical instrument for use inside the living body, a coil for supplying power to the medical instrument within the living body from outside the living body is provided to the extracorporeal unit. Herein, although the details are described later, the medical instrument includes a medicinal solution container that stores a medicinal solution, a soft portion that closes the medicinal solution container, a coil (first coil) that receives electric power from outside, a light emitting unit arranged around the soft portion and/or the medicinal solution container, etc. In addition, the extracorporeal unit includes a coil (second coil) that transmits electric power to outside. The coil of the medical instrument receives the electric power transmitted from the extracorporeal unit, and causes the light emitting unit to emit light. By the light emitting unit emitting light, the user or end-user can confirm the position of the soft portion punctured by the injection needle. Herein, from the viewpoint of power supply efficiency, after moving the extracorporeal unit to the position at which the medical instrument is implanted, it is desirable to fix so that the positions of the first coil of the medical instrument and the second coil of the extracorporeal unit do not move.

For example, it has been considered to fix the coil (second coil) portion of the extracorporeal unit to the living body which is the patient. However, in the fixing of the extracorporeal unit relative to the living body, there is a possibility of injuring the skin of the patient due to pasting surgical tape or the like directly to the living body. Herein, the medical instrument is, for example, a subcutaneous implantable port (CV port). The CV port is mainly used in chemotherapy of cancer in which the injection of a chemotherapeutic agent is performed a plurality of times for a long period. If sticking the surgical tape, etc. to the skin of the patient whose skin has already thinned from the side effects of chemotherapy, the patient is subject to great discomfort with the removal of tape and irritation of the adhesive, and thus the use of surgical tape or the like is not preferable in the medical field.

For this reason, by providing a holding portion to a power transmission unit of the extracorporeal unit, it is possible to easily carry out power supply to the medical instrument in the living body and fixing of the medical instrument from outside the living body with one hand by pinching the body surface, and further, it is possible to easily carry out the above-mentioned power supply and fixing of the medical instrument even by the patient himself/herself, a result of which it was found that the burden can be reduced on the living body during medicinal solution injection to the medicinal instrument in the living body, and based on this knowledge, arrived at completion of the present disclosure.

A medical device according to an embodiment includes: the extracorporeal unit including the power transmission unit having the second coil that transmits electric power in a non-contact manner; a power receiving unit having a first coil that receives the electric power transmitted from the power transmission unit; the implantable medical instrument for use in a living body, the implantable medical instrument including the power receiving unit; and the notification unit that is provided to at least either one of the extracorporeal unit and the medical instrument, and in the case of a relative positional relationship between the power transmission unit and the power receiving unit reaching a predetermined state along with movement of the power transmission unit, notifies of having entered said predetermined state. In the medical device, the power transmission unit includes a sheet portion assuming a plate shape, a cable that supplies electric power to the second coil, and one or more holding portions for holding the power transmission unit; and the second coil is provided inside of the sheet portion, and a hole for insertion of an injection needle is provided in the second coil.

An extracorporeal unit of the embodiment detects a predetermined location of the implantable medical instrument for use in a living body, the implantable medical instrument including a power receiving unit that receives the electric power transmitted from outside in a non-contact manner. This extracorporeal unit includes a power transmission unit having a second coil that transmits electric power in a non-contact manner, in which the power transmission unit includes a sheet portion assuming a plate shape, a cable that supplies electric power to the second coil, and one or more holding portions for holding the power transmission unit; and the second coil is provided inside of the sheet portion, and a hole for insertion of an injection needle is provided in the second coil.

A power transmission sheet of the embodiment is attachable to and detachable from the extracorporeal unit detecting a predetermined location of the implantable medical instrument for use in a living body, the implantable medical instrument including the power receiving unit that receives electric power transmitted from outside in a non-contact manner. This power transmission sheet includes a sheet portion assuming a plate shape, in which a second coil that transmits electric power in a non-contact manner is provided inside of the sheet portion, one or more holding portions for holding the sheet portion are provided on an outer side of the sheet portion, and a hole for insertion of an injection needle is provided in the second coil.

### First Embodiment

### (Configuration of Medical Device)

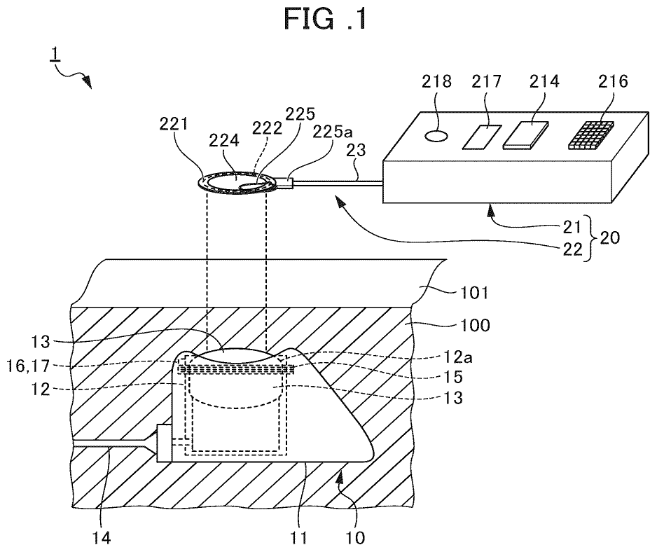

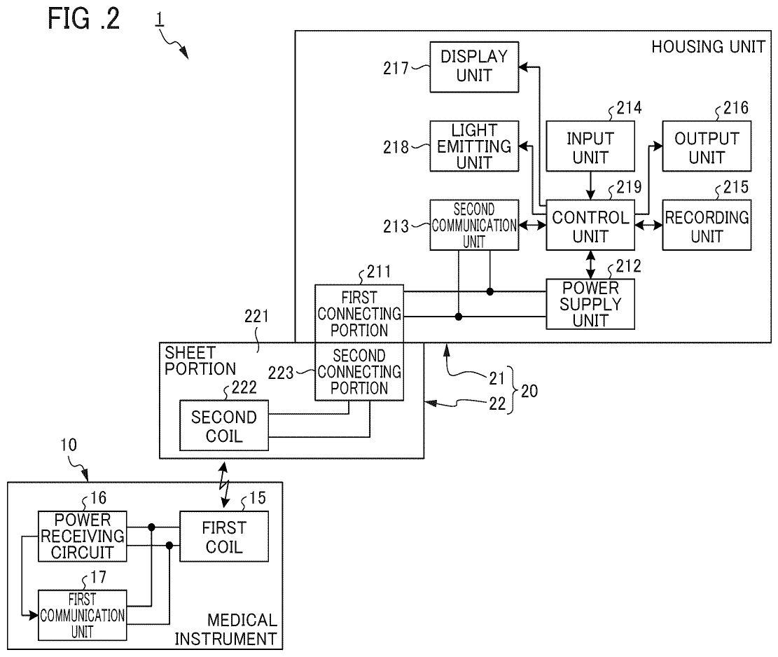

FIG. 1 is a schematic diagram showing an example of an outline configuration of a medical device according to a first embodiment. FIG. 2 is a block diagram showing an example of a functional configuration of the medical device according to the first embodiment. As shown in FIGS. 1 and 2, the medical device 1 according to the first embodiment includes an implantable medical instrument 10 for use in a living body 100, and an extracorporeal unit 20 that detects a predetermined location on the medical instrument 10. With the medical device 1 according to the first embodiment, a notification portion described later is provided to the extracorporeal unit 20.

### (Configuration of Medical Instrument)

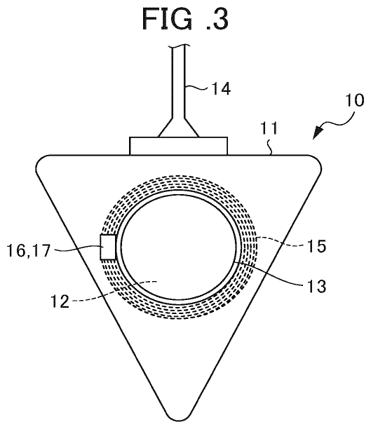

First, a detailed configuration of the medical instrument 10 will be described. FIG. 3 is a schematic diagram showing an example of an outline configuration of the medical instrument 10 constituting the medical device 1 according to the first embodiment.

The medical instrument 10, for example, is referred to as a subcutaneous implantable port (CV port). The medical instrument 10 is used by a main body portion 11 being implanted in the living body 100, as shown in FIGS. 1 to 3.

The main body portion 11 is a housing made of epoxy resin or the like, for example. The main body portion 11 includes a medicinal solution container 12, a soft portion 13, a catheter 14, a first coil 15, a power receiving circuit 16 and a first communication unit 17, as shown in FIGS. 1 to 3.

The medicinal solution container 12 is a chamber for temporarily storing the medicinal solution. The medicinal solution container 12 has a circular opening 12a toward the outside of the living body.

The soft portion 13 is referred to as a so-called septum. Then, the soft portion 13 closes the opening 12a of the medicinal solution container 12. The soft portion 13 is a soft lid (silicone diaphragm) made of, for example, silicone rubber, and is exposed from the main body portion 11. In addition, the soft portion 13 assumes a column shape. The soft portion 13 is a portion through which an injection needle for medicinal solution injection (transfusion) can be inserted from a body surface 101 of the subject (living body).

One end of the catheter 14 communicates with the medicinal solution container 12, and the other end communicates with a blood vessel (not shown). This catheter 14 transports the medicinal solution temporarily stored in the medicinal solution container 12.

The first coil 15 is formed by winding a plurality of times around an end face on the outer side of the medicinal solution container 12 in an annular shape, and is provided around the soft portion 13. This first coil 15 receives electric power sent from the power transmission portion of the extracorporeal unit 20. In addition, the first coil 15 is mainly configured from copper wire in order to reduce size and increase power supply; however, it may be configured from aluminum wire in order to further reduce weight. For example, the first coil 15 is formed so as to be on the order of 5 g. The first coil 15 is formed by molding in a form integrated with the main body portion 11.

The power receiving circuit 16 receives the electric power generated in the first coil 15 (induced electromotive force), and outputs the received electric power result to the first communication unit 17. The first coil 15 and the power receiving circuit 16 function as a power receiving unit.

The first communication unit 17 receives the received electric power result inputted from the power reception circuit 16 via the first coil 15, and transmits a radio wave to the extracorporeal unit 20. The power receiving circuit 16 and the first communication unit 17 may be integrally formed in the IC chip, or may be separately formed. It should be noted that, when a subject implanted with the medical instrument 10 in the living body uses MRI, the IC chip may be exposed to a strong magnetic field. Therefore, the medical instrument 10 may include a current detecting function for detecting a current flowing through the first coil 15, and a cut-off function such as a switch for electrically isolating the first coil 15, the power receiving circuit 16, and the first communication unit 17. According to such a configuration, when a large current equal to or greater than a predetermined threshold is detected by the current detection function, it is possible for the IC chip to isolate the current flowing through the first communication unit 17 by electrically isolating the first coil 15, the power receiving circuit 16, and the first communication unit 17 by the cut-off function.

### (Configuration of Extracorporeal Unit)

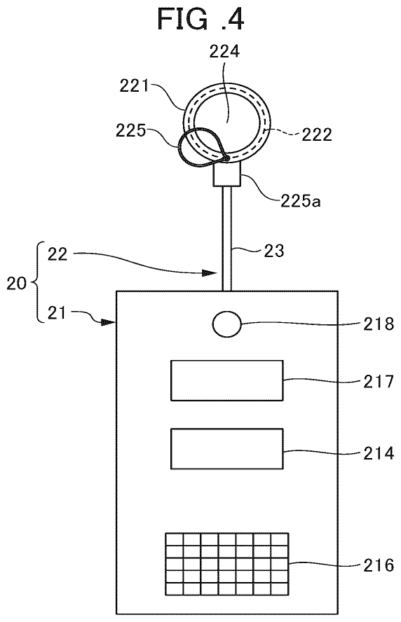

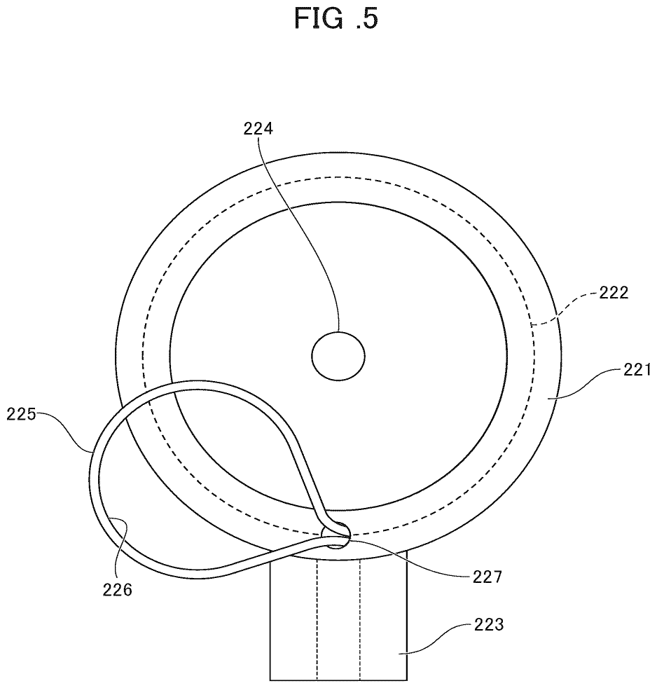

Next, an outline configuration of the extracorporeal unit 20 will be described. FIG. 4 is a schematic diagram showing an example of an outline configuration of the extracorporeal unit 20 constituting the medical device 1 according to the first embodiment. FIG. 5 is a schematic diagram showing an example of an outline configuration of the power transmission unit 22 constituting the medical device 1 according to the first embodiment.

As shown in FIGS. 1 and 2, and FIGS. 4 and 5, the extracorporeal unit 20 detects a predetermined location on the implantable medical instrument 10 for use in the living body 100. When the relative positional relationship between the first coil 15 of the medical instrument 10 and the power transmission unit 22 reaches a predetermined state, the extracorporeal unit 20 notifies that the relative positional relationship between the first coil 15 and the power transmission unit 22 has reached a predetermined state.

The extracorporeal unit 20 includes a housing unit 21 and the power transmission unit 22. It should be noted that, in the present embodiment, the power transmission sheet is included in the concept of the power transmission unit. The power transmission unit 22 includes a sheet portion 221 assuming a plate shape, a cable 23 that supplies electric power to a second coil 222, and one or more holding portions 225 for holding the power transmission unit 22. The second coil 222 is provided inside of the sheet portion 221.

The holding portion 225 is provided to at least one of the sheet portion 221 and the cable 23. Herein, the extracorporeal unit 20 in which the holding portion 225 is provided to the sheet portion 221 will be described.

As shown in FIGS. 1 and 4, the housing unit 21 and the sheet portion 221 of the power transmission unit 22 are connected via the cable 23 having a predetermined length, and electric power is supplied from the housing unit 21 to the second coil 222 of the power transmission unit 22. The second coil 222 stored within the sheet portion 221 of the power transmission unit 22 is connected to an end of the cable 23. In addition, the other end of the cable 23 is connected to the housing unit 21.

The housing unit 21 constituting the extracorporeal unit 20 accommodates a circuit board in which a power supply, IC chip, etc. are mounted inside. The housing unit 21 supplies electric power to the power transmission unit 22, and receives the received electric power result received by the power transmission unit 22. The power transmission unit 22 includes the second coil 222 that transmits electric power in a non-contact manner.

The housing unit 21 includes a first connecting portion 211, a power supply unit 212, a second communication unit 213, an input unit 214, a recording unit 215, an output unit 216, a display unit 217, a light emitting unit 218, and a control unit 219. In the first embodiment, at least one of the output unit 216, the display unit 217, and the light emitting unit 218 function as a notification unit.

The other end of the cable 23 is electrically connected to the first connecting portion 211, and the electric power inputted from the power supply unit 212 is supplied to the second coil 222 via the cable 23. The first connecting portion 211 is configured using a female electrical coupler.

The power supply unit 212 supplies electric power to the power transmission unit 22 via the first connecting portion 211 under the control of the control unit 219. The power supply unit 212 uses a battery, a booster circuit, and other components.

The second communication unit 213 outputs the received electric power result (signal) received by the power transmission unit 22 via the first connecting portion 211 to the control unit 219 under the control of the control unit 219.

The input unit 214 receives an input of an operation from the user. The input unit 214 inputs values based on the operations of the user to the control unit 219. In addition, the input unit 214 is realized by using buttons, switches, a touch panel, and other components. Here, the user refers to any one of a medical doctor, a nurse, a caregiver, and a patient himself/herself as a subject.

The recording unit 215 records information and various programs executed by the extracorporeal unit 20. The recording unit 215 is realized using volatile memory and nonvolatile memory.

The output unit 216 outputs sound under the control of the control unit 219. The output unit 216, for example, is realized using a speaker or the like. In addition, in the case of a relative positional relationship between the power transmission unit 22 and the power receiving unit of the medical instrument 10 having reached a predetermined state along with the movement of the power transmission unit 22, the output unit 216 can notify of entering this predetermined state by sound.

The display unit 217 displays predetermined information under the control of the control unit 219. The display unit 217 is realized by using, for example, a liquid crystal or an organic EL (Electro Luminescence). In addition, in the case of a relative positional relationship between the power transmission unit 22 and the power receiving unit of the medical instrument 10 having reached a predetermined state along with the movement of the power transmission unit 22, the display unit 217 can notify of entering this predetermined state by display.

The light emitting unit 218 emits light under the control of the control unit 219. The light emitting unit 218 is realized by using LEDs (Light Emitting Diode). In addition, in the case of a relative positional relationship between the power transmission unit 22 and the power receiving unit of the medical instrument 10 having reached a predetermined state along with the movement of the power transmission unit 22, the light emitting unit 218 can notify of entering this predetermined state by light emission.

The control unit 219 controls the respective portions constituting the extracorporeal unit 20. When the input unit 214 is pressed, the control unit 219 controls the power supply unit 212 based on the inputted value from the input unit 214, and the power supply unit 212 supplies electric power to the power transmission unit 22 via the first connecting portion 211 based on the control by the control unit 219. Furthermore, when the received power result is inputted from the second communication unit 213, the control unit 219 causes the light emitting unit 218 to emit light so as to notify that the relative positional relationship between the power transmission unit 22 and the medical instrument 10 has reached a predetermined state. More specifically, the control unit 219 causes the light emitting unit 218 to emit light so as to notify that the relative positional relationship with the medical instrument 10 has reached a predetermined state, for example, a state in which the second coil 222 of the power transmission unit 22 is located on the first coil 15 of the medical instrument 10, based on the electric power of the received power result from the second communication unit 213. The control unit 219 is realized using memory and a processor having hardware such as a CPU (Central Processing Unit).

The power transmission unit 22 constituting the extracorporeal unit 20 has a function of transmitting electric power in a non-contact manner. As shown in FIG. 5, the second coil 222 for transmitting electric power is provided inside of the sheet portion 221 of the power transmission unit 22, and a hole 224 for insertion of an injection needle is provided in the second coil 222. Furthermore, the sheet portion 221 includes the second connecting portion 223 that electrically connects the second coil 222 and the power supply unit 212 of the housing unit 21 via the cable 23. In addition, one or more of the holding portions 225 for holding the power transmission unit 22 are provided on an outer side of the sheet portion 221. It should be noted that, since it is necessary to prevent the soft portion 13 from being removed when the injection needle is pierced, the size of the hole 224 is preferably formed to be slightly smaller than the size of the soft portion 13.

The sheet portion 221 is equipped with the second coil 222 inside thereof, and has a ring shape, e.g., annular shape. The sheet portion 221 is realized using, for example, a nonwoven fabric or the like. Furthermore, the sheet portion 221 includes two sheets of nonwoven fabric sandwiching the second coil 222 and the second connecting portion 223.

The second coil 222 has an annular or substantially annular shape, and is provided inside the sheet portion 221. The second coil 222 generates a magnetic flux in response to the electric power inputted from the housing unit 21 via the second connecting portion 223.

One end of the cable 23 is electrically connected to the second connecting portion 223, whereby the second coil 222 and the power supply unit 212 of the housing unit 21 are electrically connected. The second connecting portion 223 includes, for example, a male electrical coupler, as shown in FIG. 5.

The hole 224 is provided in the second coil 222 (sheet portion 221). For example, the hole 224 is provided at the center of the second coil 222. The hole 224 allows for the insertion and removal of the injection needle. It should be noted that the inner diameter of the second coil 222 and the diameter of the hole 224 can be appropriately changed in accordance with the diameter of the soft portion 13.

The holding portion 225 is a portion which is held by the end-user using the extracorporeal unit 20. The end-user can easily hold the holding portion 225 by a fingertip portion including fingers, a wrist portion or an arm portion. The holding portion 225 is provided to one main surface of the sheet portion 221.

Herein, a specific usage example of the holding portion 225 will be described. Generally, since puncturing with the injection needle is done with the dominant hand, in the case of the dominant hand being the right hand, the operation of the extracorporeal unit 20 is performed with the left hand which is the non-dominant hand. The end-user pinches the medical instrument 10 implanted inside a living body from above the skin of the patient with the thumb and index finger of the left hand. The positional relationship of the left hand which is the non-dominant hand and the medical instrument 10 implanted in the living body is fixed through the puncture operation. In this state, by fixing the sheet portion 221 equipped with the second coil 222 to the non-dominant hand, the medical instrument 10 is fixed with one hand, and the positional relationship between the second coil 222 in the sheet portion 221 and the implanted medical instrument 10 is fixed, and thus the stable non-contact power supply efficiency can be maintained. At this time, it is possible to easily hold the holding portion 225 with fingers of the left hand which is not used in fixing of the medical instrument 10. In addition, the holding portion 225 may be held with the entire palm of the left hand, without using fingers.

In addition, the cable 23 connecting the second coil 222 and the housing unit 21 is configured from heavy and thick diameter cable due to EMC (Electromagnetic Compatibility) requirements, and may be inflexible. As mentioned above, from the viewpoint of power supply efficiency, after moving the second coil 222 to the position at which the medical instrument 10 is implanted, it is desirable to fix so that the positions of the first coil 15 of the medical instrument 10 and the second coil 222 do not move, and thus it is necessary to control movement of the cable 23. In the present example, since the end-user pinches the medical instrument 10 implanted in the living body from above the skin of the patient with the thumb and index finger of their non-dominant hand (for example, left hand), for example, and holds the holding portion 225 with their middle finger or the like, it is possible to firmly hold and fix the second coil 222 connected to one end of the cable 23 with a non-dominant hand, and thus movement of the cable 23 can be controlled.

In addition, as shown in FIG. 5, for example, the sheet portion 221 may further include a coupling hole 227 that is provided to one main surface of the sheet portion 221 and that couples the sheet portion 221 and the holding portion 225, and a part of the holding portion 225 may be inserted in the coupling hole 227. In this case, after inserting a part of the holding portion 225 into the coupling hole 227, an adhesive may be filled into the coupling hole 227 to adhere the sheet portion 221 and the holding portion 225.

By the end-user holding the holding portion 225, the end-user can easily perform movement of the power transmission unit 22 to search for the location of the medical instrument 10 implanted within the living body 100 from the extracorporeal unit 20, while stably holding the sheet portion 221. In addition, in the case of the relative positional relationship between the power transmission unit 22 of the extracorporeal unit 20 and the power receiving unit of the medical instrument 10 reaching a predetermined state, the end-user ends movement of the power transmission unit 22, and can easily maintain stable holding of the sheet portion 221 to the body surface 101 of the living body 100.

End-user holding the holding portion 225 refers to any one of a medical doctor, a nurse, a caregiver, and a patient himself/herself as a subject, and may be the same as user. In particular, when the end-user is the patient, movement of the power transmission unit 22 for searching for the medical instrument 10, maintaining of holding of the power transmission unit 22 for sustaining the predetermined positional relationship between the power transmission unit 22 and the power receiving unit of the medical instrument 10, and puncturing of the injection needle to the living body 100 for injecting the medicinal solution into the living body 100 can be easily carried out by the patient himself/herself.

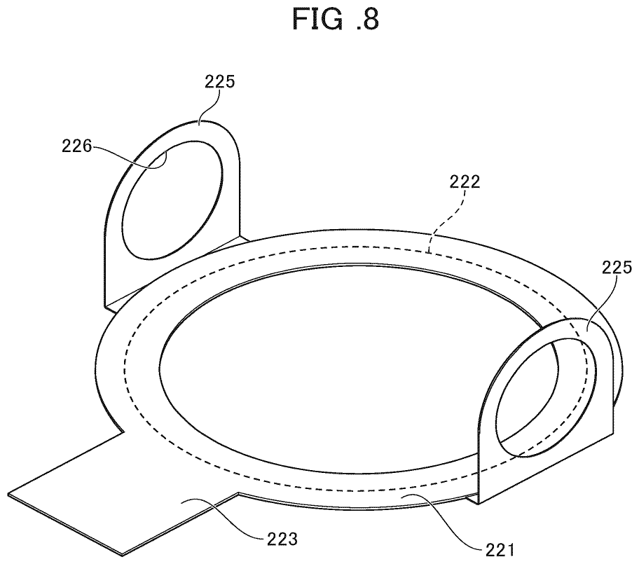

In addition, as shown in FIG. 5 and FIG. 8 described later, the holding portion 225 preferably has a through hole 226. The end-user can easily hold the holding portion 225 by hand by inserting a finger or the like into the through hole 226. In the case of the through hole 226 being circular, the hole diameter of the through hole 226 is on the order of 80 to 90 mm, for example.

Such a holding portion 225 is preferably provided to stand on one main surface of the sheet portion 221, as shown in FIG. 8, and is more preferably provided to stand on one main surface of the sheet portion 221 during use. If the holding portion 225 includes the through hole 226, during use, the end-user can easily hold the holding portion 225 by placing a finger in the holding portion 225 in a state provided standing from one main surface of the sheet portion 221. For this reason, the patient himself/herself can easily carry out a series of operations including performing movement of the power transmission unit 22, pinching of the medical instrument 10 from above the skin, and maintaining of the holding of the power transmission unit 22 with one hand, and further performing puncturing of the injection needle with the other hand.

In addition, the holding portion 225 may be a rigid body as in FIG. 8, or be soft or flexible as in the above-described drawing.

Next, a sequence of detecting the position of the medical instrument 10 will be described. Herein, a case of the user and the end-user being the same will be described. The user causing electric power to be supplied to the second coil 222 via the cable 23 from the power supply unit 212 of the housing unit 21, by pressing down the input unit 214 of the extracorporeal unit 20. Then, the user holds the holding portion 225 of the extracorporeal unit 20, and makes the power transmission unit 22 of the extracorporeal unit 20 approach the medical instrument 10 implanted in the living body 100, while searching for this. In this case, the second coil 222 causes a magnetic field to be generated by the electric power being supplied from the power supply unit 212 via the cable 23.

Next, the user causes the sheet portion 221 of the extracorporeal unit 20 to further approach the medical instrument 10, while holding the holding portion 225 of the extracorporeal unit 20. Under this condition, in the case of the relative positional relationship between the first coil 15 of the medical instrument 10 and the second coil 222 of the extracorporeal unit 20 reaching a predetermined position, the light emitting unit 218 notifies the user by performing light emission indicating as having reached the predetermined location on the medical instrument 10. In this case, in the case of the positional relationship between the first coil 15 of the medical instrument 10 and the second coil 222 of the extracorporeal unit 20 reaching positions overlapping each other in the vertical direction, the medical device 1 notifies the user by the light emitting unit 218 emitting light. As a result, the user can easily confirm the position of the soft portion 13 of the implantable medical instrument 10 for use in the living body 100. More specifically, the user can grasp that the soft portion 13 is located under the hole 224 at the location where the light emitting unit 218 emitted light.

It should be noted that, when the relative positional relationship between the first coil 15 of the medical instrument 10 and the second coil 222 of the extracorporeal unit 20 have reached a predetermined position, the control unit 219 may notify the user by not only causing the light emitting unit 218 to emit light, but also causing the output unit 216 to output sound. Furthermore, the control unit 219 may notify the user by causing the display unit 217 to display information indicating that the second coil 222 has reached the soft portion 13 of the medical instrument 10.

Thereafter, the user pinches the medical instrument 10 implanted in the living body from above the skin with the fingers of one hand holding the holding portion 225, while maintaining the predetermined positional relationship between the first coil 15 of the medical instrument 10 and the second coil 222 of the extracorporeal unit 20. In this state, the injection needle is inserted in the hole 224 formed on the inner side of the power transmission unit 22 with the other hand at a mark provided thereon. It is thereby possible for the user to insert the injection needle into the soft portion 13 without losing the position of the soft portion 13 of the medical instrument 10, while holding the holding portion 225 of the extracorporeal unit 20, and it is possible to easily inject the medicinal solution into the medicinal solution container 12.

In addition, in the case of the user and end-user being different, the user operates the housing unit 21 of the extracorporeal unit 20, and the end-user operates the power transmission unit 22 of the extracorporeal unit 20. Operation of the injection needle may be by the user or by the end-user.

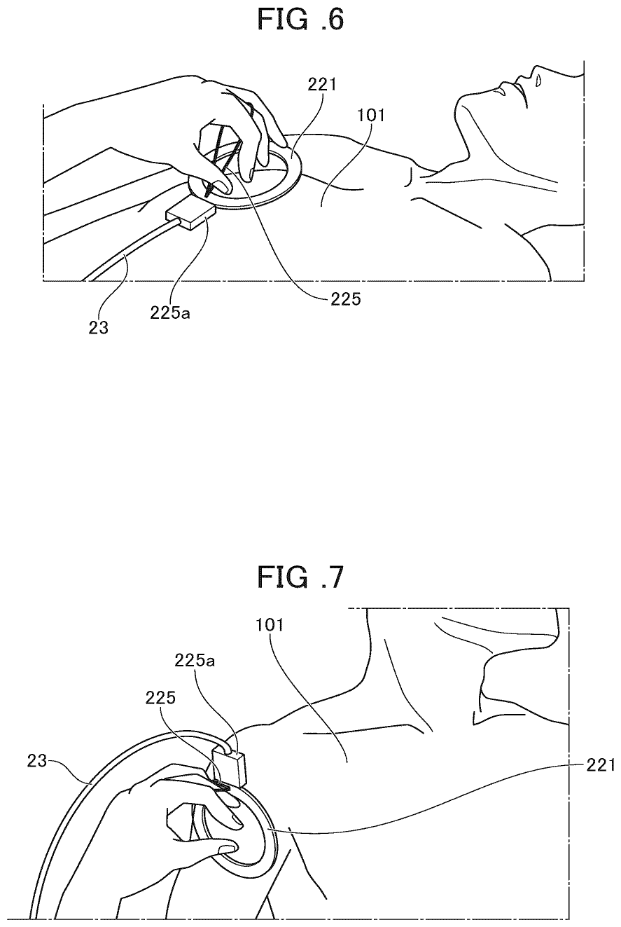

In addition, depending on where the medical instrument 10 is implanted in the living body 100 which is the patient, the state of fixing the power transmission unit 22 incorporating the second coil 222 will differ. In the case of the medical instrument 10 being implanted in the chest of the patient, the patient receives the puncture of the injection needle in a state laying down on his/her back on a bed or the like. In this case, as shown in FIG. 6, the end-user pinches the medical instrument 10 implanted in the living body 100 from above the skin of the patient with the thumb and index finger of the non-dominant hand (for example, left hand), and holds the holding portion 225 by the middle finger, for example, of the non-dominant hand. The power transmission unit 22 is fixed substantially parallel to the ground surface. In addition, in the case of the medical instrument 10 being implanted in the arm of the patient, the patient receives the puncture of the injection needle in a state seated on a chair, bed or the like. In this case, as shown in FIG. 7, the end-user pinches the medical instrument 10 implanted in the living body 100 from above the skin of the patient with the thumb and index finger of the non-dominant hand (for example, left hand), and holds the holding portion 225 by the middle finger, for example, of the non-dominant hand. The power transmission unit 22 is fixed substantially perpendicular to the ground surface.

According to the first embodiment described above, in the case of the relative positional relationship between the first coil 15 of the medical instrument 10 and the second coil 222 of the extracorporeal unit 20 reaching a predetermined position, while holding the holding portion 225 of the extracorporeal unit 20, since it is notified that the relative positional relationship between the first coil 15 of the medical instrument 10 and the second coil 222 of the extracorporeal unit 20 reached the predetermined position by the light emitting unit 218 emitting light, it is possible to easily confirm the position of the soft portion 13 of the implantable medical instrument 10 for use in the living body 100. Furthermore, fixing of the power transmission unit 22 to the body surface 101 using surgical tape or the like becomes unnecessary, due to being able to puncture the injection needle to the living body 100, while easily holding the holding portion 225 of the power transmission unit 22. For this reason, it is possible to avoid damage to the skin of the patient or discomfort to the patient from directly applying surgical tape or the like to the body surface 101. In this way, it is possible to reduce the burden on the living body 100 during medicinal solution injection to the medical instrument 10 in the living body 100.

In addition, one hand of the end-user and the power transmission unit 22 are fixed by holding the holding portion 225. When the end-user is the patient, since one hand of the end-user is integrated with the power transmission unit 22, by performing with one hand the movement of the power transmission unit 22 for searching for the first coil 15 of the medical instrument 10, the maintaining of the holding of the power transmission unit 22 for sustaining the predetermined positional relationship between the second coil 222 of the power transmission unit 22 and the first coil 15 of the medical instrument 10, and the pinching of the medical instrument 10 from above the skin, and performing with the other hand the puncture of the injection needle for injecting a medicinal solution into the living body 100, the patient himself/herself can easily carry out a series of operations.

In addition, since the end-user pinches the medical instrument 10 implanted in the living body from above the skin of the patient with the thumb and index finger of the non-dominant hand (for example, left hand), and holds the holding portion 225 with the middle finger, for example, of the non-dominant hand, it is possible to fix so that the positions of the first coil 15 of the medical instrument and the second coil 222 of the extracorporeal unit 20 do not move.

In addition, when the power transmission unit 22 of the extracorporeal unit 20 is a detachable configuration relative to the housing unit 21 via the cable 23, since it is possible to reuse the housing unit 21 even if for another subject, the cost upon introducing the medical device 1 can be reduced.

It should be noted that, although the above description illustrates an example providing one holding portion 225, it is sufficient so long as there is at least one holding portion 225, and a plurality of holding portions 225 may be provided, as shown in FIG. 8. The sizes of the plurality of holding portions 225 may all be the same, part thereof may be different, or all may be entirely different.

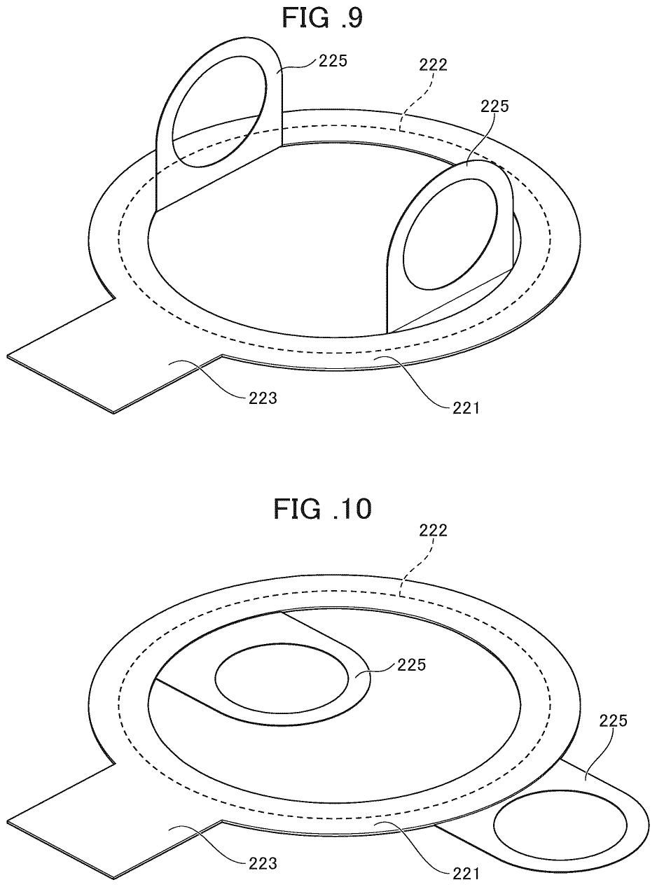

In addition, the holding portion 225 may be provided on a main surface of the sheet portion 221 as shown in FIG. 5 described above, may be provided at the outer edge of the sheet portion 221 as shown in FIG. 8, or may be provided at the inner edge of the sheet portion 221 as shown in FIG. 9. In addition, the holding portion 225 may be provided to be standing relative to the main surface of the sheet portion 221 as shown in FIGS. 8 and 9, or may be provided to extend along the main surface of the sheet portion 221. For example, as shown in FIG. 10, the holding portion 225 provided at the outer edge of the sheet portion 221 may extend at the outer side of the sheet portion 221 along the main surface of the sheet portion 221. In addition, as shown in FIG. 10, the holding portion 225 provided at the inner edge of the sheet portion 221 may extend at the inner side of the sheet portion 221 along the main surface of the sheet portion 221.

In addition, although FIG. 5 illustrates an example in which the holding portion 225 is provided to the sheet portion 221, the holding portion 225 may be provided to the cable 23 and/or a holding portion 225a described later, as shown in FIG. 11.

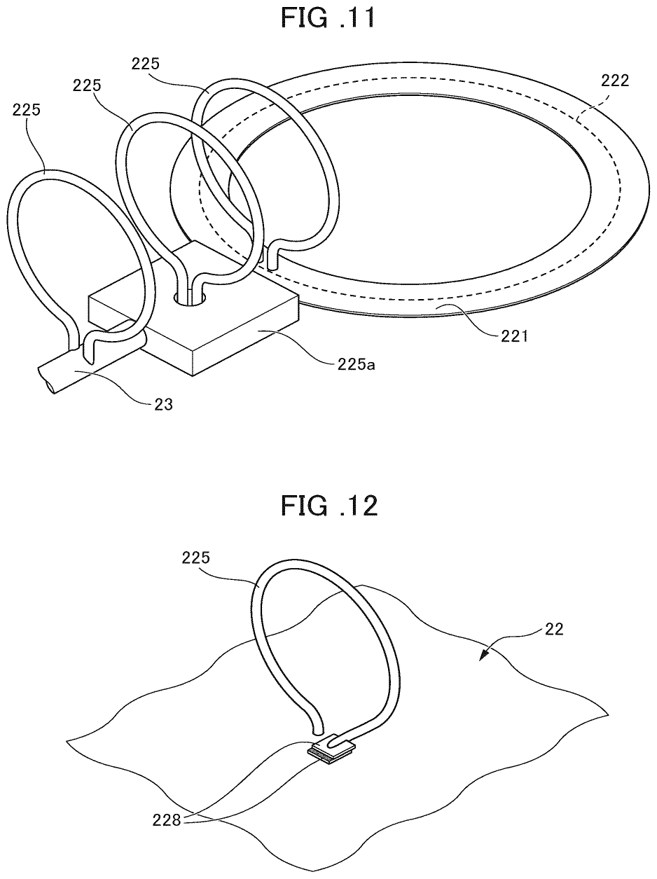

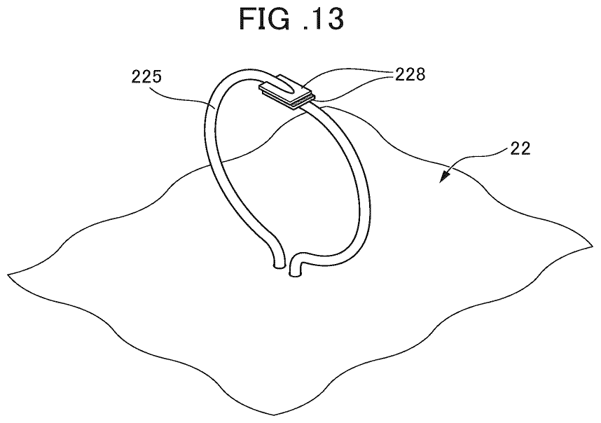

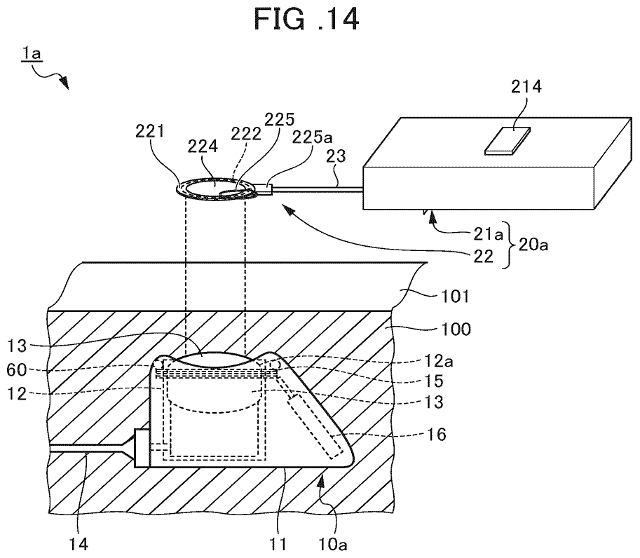

In addition, the configuration of the holding portion 225 may be a ring shape as shown in FIG. 5. In addition, as shown in FIG. 12, the holding portion 225 may be a configuration which is linear, in which one end of the holding portion is fixed to the power transmission unit 22, and a mounting portion 228 provided to the other end of the holding portion is detachably mounted to the power transmission unit 22. In addition, as shown in FIG. 13, the holding portion 225 may be a configuration with a plurality of linear shapes, in which one end of the plurality of holding portions is fixed to the power transmission unit 22, and mounting portions 228 provided to the other end of the plurality of holding portions are detachably mounted to each other. The mounting portions 228 are preferably hook-and-loop fasteners, buttons or metal fittings.

In addition, although the holding portion 225 has been described above, the holding portion may be a holding portion 225a shown in FIG. 1, etc. In this case, the medical device 1 may include only the holding portion 225, may include only the holding portion 225a, or may include both the holding portion 225 and the holding portion 225a. In addition, the holding portion 225 may be provided to the holding portion 225a.

Relative to the holding portion 225 held by the fingertip portion including fingers, wrist portion of arm portion, the holding portion 225a is mainly gripped by the fingertip portion including fingers. As shown in FIG. 1, the holding portion 225a is provided between the sheet portion 221 and the cable 23. From the viewpoint of easily gripping the holding portion 225a by the fingertip portion, the holding portion 225a is preferably a rigid body. In addition, from the same viewpoint, the shape of the holding portion 225a is preferably a polyhedron.

In addition, the holding portion 225a may have the function of a connector. In this case, the second coil 222 housed inside of the sheet portion 221 of the power transmission unit 22 is connected to one side of the cable 23 via the holding portion 225a. One side of the cable 23 is electrically connected to the second connecting portion 223 via the holding portion 225a, whereby the second coil 222 and the power supply unit 212 of the housing unit 21 are electrically connected.

### Second Embodiment

Next, a second embodiment will be described. In the aforementioned first embodiment, although notification is made by providing the notification unit to the extracorporeal unit, the second embodiment notifies by providing the notification unit to the medical instrument. It should be noted that the same reference symbols are attached to configurations identical to the medical device 1 according to the aforementioned first embodiment, and detailed descriptions thereof will be omitted.

### (Configuration of Medical Device)

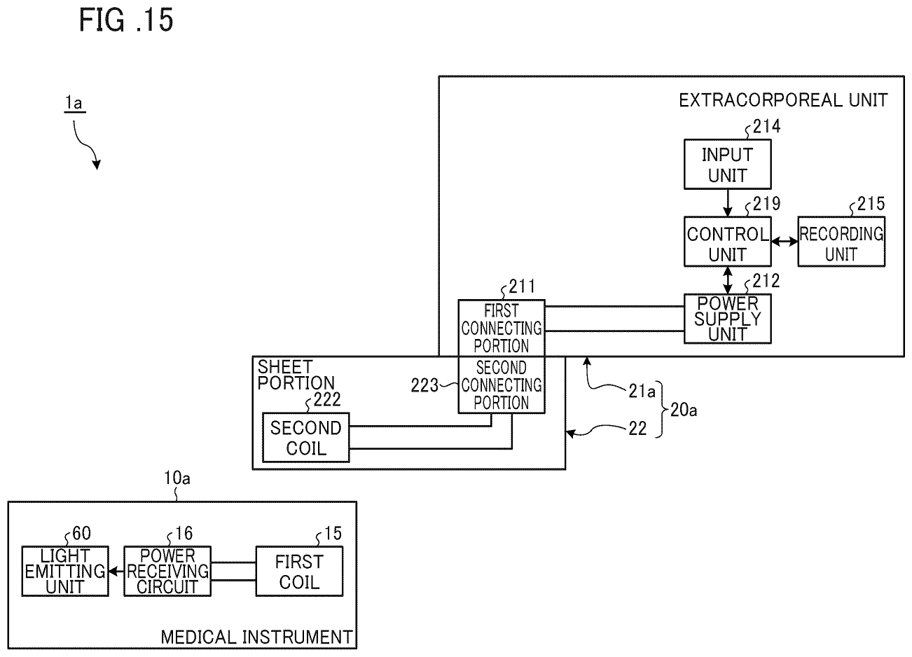

FIG. 14 is a schematic diagram showing an example of an outline configuration of a medical device according to a second embodiment. FIG. 15 is a block diagram showing an example of the functional configuration of the medical device according to the second embodiment. As shown in FIGS. 14 and 15, a medical device 1a according to the second embodiment includes an implantable medical instrument 10a for use in the living body 100, and an extracorporeal unit 20a that detects a predetermined location of the medical instrument 10a. With the medical device 1a according to the second embodiment, the notification unit described later is provided to the medical instrument 10a. In addition, herein, both the holding portion 225 and the holding portion 225a are provided; however, it is sufficient if at least either one of the holding portion 225 and the holding portion 225a is provided.

### (Configuration of Medical Instrument)

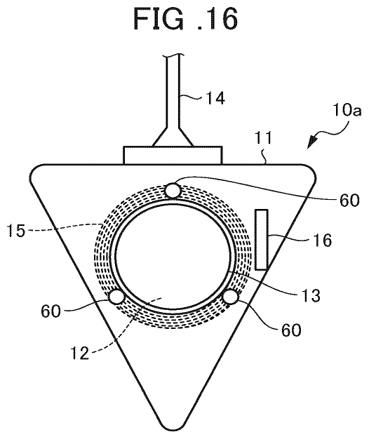

First, the detailed configuration of the medical instrument 10a will be described. FIG. 16 is a schematic diagram showing an example of an outline configuration of the medical instrument 10a constituting the medical device 1a according to the second embodiment.

As shown in FIGS. 14 to 16, the medical instrument 10a includes the light emitting unit 60 as the notification unit.

The power receiving unit of the medical instrument 10a includes the first coil 15 and the power receiving circuit 16. The first coil 15 receives electric power transmitted from the power transmission unit 22 of the extracorporeal unit 20a. The power receiving circuit 16 transmits the electric power (induced electromotive force) received by the first coil 15 to the light emitting unit 60, which is the notification unit. Furthermore, the power receiving circuit 16 converts the electric power received by the first coil 15 to a predetermined electric power, and transmits the converted electric power to the light emitting unit 60.

The implantable medical instrument 10a for use in a living body has a power receiving unit. In the case of the relative positional relationship between the power transmission unit 22 and the power receiving unit reaching a predetermined state along with the movement of the power transmission unit 22 provided to the extracorporeal unit 20a, the light emitting unit 60 notifies of having entered this predetermined state by emitting light using the electric power received by the power receiving unit.

A plurality of the light emitting units 60 are arranged around the soft portion 13. For example, as shown in FIG. 16, the plurality of light emitting units 60 are arranged at three locations around the soft portion 13 in an annular shape every 120 degrees along the outer edge of the soft portion 13. The light emitting unit 60 emits light in response to the electric power received by the power receiving circuit 16. For example, the light emitting units 60 each include an LED. More specifically, it is preferable for the light emitting unit 60 to use an LED having high directivity. More specifically, it is preferable that the light emitting units 60 each use a red LED that emits light in a red wavelength band when transmitting through a living body such as a subject.

### (Configuration of Extracorporeal Unit)

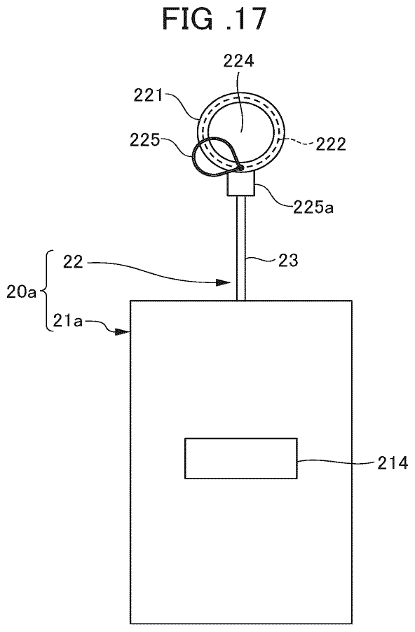

Next, the configuration of the extracorporeal unit 20a will be described. FIG. 17 is a schematic diagram showing an example of an outline configuration of the extracorporeal unit 20a constituting the medical device 1a according to the second embodiment. As shown in FIGS. 14, 15 and 17, the extracorporeal unit 20a includes a housing unit 21a and the power transmission unit 22. It should be noted that, in the present embodiment, the power transmission sheet is included in the concept of the power transmission unit. In the extracorporeal unit 20a, the second communication unit 213, the output unit 216, the display unit 217 and the light emitting unit 218 are removed from the configuration of the aforementioned extracorporeal unit 20.

The second coil 222 assumes an annular shape, and generates a magnetic flux in response to the electric power inputted from the power supply unit 212, and transmits the electric power to the first coil 15 of the implantable medical instrument 10a for use in the living body 100. In addition, the electric power supplied by the second coil 222 is of an extent enabling the light emitting unit 60 to emit light, even if the second coil 222 and the first coil 15 are at a predetermined distance, e.g., a distance on the order of tens of centimeters.

The medical device 1a configured in this way supplies electric power from the extracorporeal unit 20a to the medical instrument 10a, and notifies the location at which the medical instrument 10a is implanted by a plurality of the light emitting units 60 in the medical instrument 10a emitting light. It is thereby possible to intuitively grasp the position and center of the soft portion 13 of the medical instrument 10a from outside of the subject.

According to the second embodiment described above, due to notifying by the plurality of light emitting units 60 of the medical instrument 10a emitting light, it is possible to intuitively grasp the position of the soft portion 13 in the medical instrument 10a from outside of the subject.

It should be noted that the first embodiment shows an example in which the notification unit is provided to the extracorporeal unit used outside of a living body, and the second embodiment shows an example in which the notification unit is provided to the implantable medical instrument for use in a living body; however, the notification unit may be provided to both the extracorporeal unit and the medical instrument.

While some of the exemplary embodiments of the present application have been described in detail based on the drawings, these are illustrative, and it is possible to implement the present invention in other forms which have been subjected to various modifications and improvements based on the knowledge of those skilled in the art, including the aspects described in the section of disclosure of the present invention.

### EXPLANATION OF REFERENCE NUMERALS

1, 1a medical device
10, 10a medical instrument
11 main body portion
12 medicinal solution container
12a opening
13 soft portion
14 catheter
15 first coil
16 power receiving circuit
17 first communication unit
20, 20a extracorporeal unit
21, 21a housing unit
22 power transmission unit
23 cable
60 light emitting unit
100 living body
101 body surface
211 first connecting portion
212 power supply unit
213 second communication unit
214 input unit
215 recording unit
216 output unit
217 display unit
218 light emitting unit
219 control unit
221 sheet portion
222 second coil
223 second connecting portion
224 hole
225, 225a holding portion
226 through hole
227 coupling hole
228 mounting portion

1. A medical device comprising:
an extracorporeal unit including a power transmission unit having a second coil that transmits electric power in a non-contact manner;
a power receiving unit having a first coil that receives the electric power transmitted from the power transmission unit;
an implantable medical instrument for use in a living body, the implantable medical instrument including the power receiving unit; and
a notification unit that is provided to at least either one of the extracorporeal unit and the implantable medical instrument, and in a case of a relative positional relationship between the power transmission unit and the power receiving unit reaching a predetermined state along with movement of the power transmission unit, notifies of having entered said predetermined state, wherein
the power transmission unit includes a sheet portion of plate shape, a cable supplying electric power to the second coil, and one or more holding portions for holding the power transmission unit, and
the second coil is provided inside of the sheet portion, and a hole for insertion of an injection needle is provided in the second coil.

2. The medical device according to claim 1, wherein the holding portion is provided to at least either of the sheet portion and the cable.

3. The medical device according to claim 1, wherein the holding portion is provided to one main surface of the sheet portion.

4. The medical device according to claim 1, wherein the sheet portion further includes a coupling hole that is provided to one main surface of the sheet portion and that couples the sheet portion and the holding portion, and
a part of the holding portion is inserted in the coupling hole.

5. The medical device according to claim 1, wherein the holding portion is provided at an outer edge of the sheet portion.

6. The medical device according to claim 1, wherein the holding portion is provided at an inner edge of the sheet portion.

7. The medical device according to claim 1, wherein the holding portion is stood on one main surface of the sheet portion.

8. The medical device according to claim 1, wherein the holding portion includes a through hole.

9. The medical device according to claim 1, wherein the holding portion is provided between the sheet portion and the cable.

10. An extracorporeal unit detecting a predetermined location of an implantable medical instrument for use in a living body, the implantable medical instrument including a power receiving unit that receives electric power transmitted from outside in a non-contact manner, the extracorporeal unit comprising:
a power transmission unit including a second coil that transmits electric power in a non-contact manner, wherein
the power transmission unit includes a sheet portion of plate shape, a cable supplying electric power to the second coil, and one or more holding portions for holding the power transmission unit, and
the second coil is provided inside of the sheet portion, and a hole for insertion of an injection needle is provided in the second coil.

11. A power transmission sheet attachable to and detachable from an extracorporeal unit that detects a predetermined location of an implantable medical instrument for use in a living body, the implantable medical instrument including a power receiving unit that receives electric power transmitted from outside in a non-contact manner, wherein
the power transmission sheet includes a sheet portion of plate shape, and
a second coil that transmits electric power in a non-contact manner is provided inside of the sheet portion, one or more holding portions for holding the sheet portion are provided on an outer side of the sheet portion, and a hole for insertion of an injection needle is provided in the second coil.

12. The medical device according to claim 2, wherein the holding portion includes a through hole.

13. The medical device according to claim 3, wherein the holding portion includes a through hole.

14. The medical device according to claim 4, wherein the holding portion includes a through hole.

15. The medical device according to claim 5, wherein the holding portion includes a through hole.
